(19) United States
(12) Patent Application Publication
(10) Pub. No.: US 2017/0266052 A1
(43) Pub. Date: Sep. 21, 2017

(54) SYSTEM FOR MEASURING AND MANUFACTURING COMPRESSION GARMENTS

(71) Applicants: Fredrick Ellis Levine, Cupertino, CA (US); Neil Seth Levine, San Francisco, CA (US)

(72) Inventors: Fredrick Ellis Levine, Cupertino, CA (US); Neil Seth Levine, San Francisco, CA (US)

(21) Appl. No.: 15/434,377

(22) Filed: Feb. 16, 2017

Related U.S. Application Data
(60) Provisional application No. 62/309,571, filed on Mar. 17, 2016.

(51) Int. Cl. A61F 13/08; A61H 1/00; G01L 5/16; A41B 11/02

(52) U.S. Cl. CPC A61F 13/08; A41B 11/02; A61H 1/008; G01L 5/16; A61H 2209/00

(57) ABSTRACT

A system for accurately measuring compression garments in laboratory and manufacturing environments provides measurements for industry-standard sizes and individually customized garments and can use the measurements to modify the programming of a manufacturing system to alter the compression parameters of subsequently manufactured garments. The system includes a support structure and a plurality of sensor units at intervals along the support structure with each sensor unit extended circumferentially around the support structure to define a three-dimensional simulated anatomical form circumferentially stretching the compression garment upon insertion of the assembly. Each sensor unit has a pressure sensor measuring the pressure exerted by the compression garment on the sensor unit after insertion.

### RELATED APPLICATION

The present application is based on and claims priority to the Applicant's U.S. Provisional Patent Application 62/309,571, entitled “System for Measuring and Manufacturing Compression Garments,” filed on Mar. 17, 2016.

### BACKGROUND OF THE INVENTION

Field of the Invention

The present invention relates generally to the field of compression garments. More particularly, the present invention is a system for accurately measuring graduated compression garments in both laboratory and manufacturing environments, and using the acquired measurement data to modify the programming of a manufacturing system.

Background of the Invention

The two devices most widely used by both industry and regulatory agencies for measurement and certification of compression garments were developed in the late 1970's. The HATRA tester, developed by Derek Peat while working at the Hosiery and Allied Trades Research Association in Nottingham, England, is referenced in British Standards BS 6612:1985 (Specification for graduated compression hosiery,) BS 7563:1999 (Specification for non-prescriptive graduated support hosiery) and BS 7672:1993 (Specification for compression, stiffness and labelling of anti-embolism hosiery.) European manufacturers and certifiers also use the HOSY tester, created by researchers at the Hohenstein Institute in Bönnigheim, Germany. Use of the HOSY system is required by German Standard RAL-GZ 387/1 (Medical Compression Hosiery Quality Assurance.) Compression certification in the United States is typically performed on a HATRA machine, although there is no formal US standard for compression garment measurement and certification. Additionally, a third device, the medical stocking tester (MST) distributed by Swisslastic AG, is sometimes used by researchers and manufacturers, and will be discussed here.

Both the HATRA and HOSY devices measure the tension of a fabric when a compression garment is stretched in two dimensions, rather than over a three-dimensional form. The pressure exerted by a garment is not measured directly, but is calculated from the fabric tension measurements using the Young-Laplace partial differential equation, simplified to express the relationship of tension to pressure in a cylinder.

Shown in FIG. 1 in a simplified diagram, the HATRA machine consists of a fixed base 101 to which is attached a stationary support bar 102. A movable support bar 103, is connected to the stationary support bar at pivot 104. An adjustment bar 105 is attached to both support bars. The separation 106 between the movable support bar and the stationary support bar is determined by a removable bolt 107 placed in one of the available holes 108 in the adjustment bar.

Two adjustable silhouette profiles are attached to the stationary support bar, one for the calf 109 and one for the thigh 110. By changing the attachment positions of the silhouette profiles using removable bolts 111, placed in one of a series of available mounting holes 112, the girth measurements of the machine may be varied to simulate a range of limb sizes. Further variations may be achieved by substituting silhouette profiles with differing dimensions. A foot support 113 is provided to support and stretch the foot of a leg garment, and aid in the correct placement of the garment. An adjustment lever 114 allows the separation of the support bars to be reduced to facilitate initial garment placement without disturbing the settings of the various adjustments.

To measure a garment on the HATRA machine, the adjustment lever 114 is lowered to reduce the support bar separation. The garment is drawn over the foot support 113 and along the support bars 102 and 103, and clamped at the upper end of the garment with clamps 115. The heel of the garment may be aligned with pivot 104 to aid in producing repeatable measurements. The adjustment lever 114 may be repeatedly raised and lowered to allow the garment fabric to distribute evenly on both sides of the support bars. When the garment fabric is correctly positioned, the adjustment lever 114 is raised to place the support bars in position to stretch the garment fabric. FIG. 2. Illustrates a sock 201 being measured on a HATRA machine.

Continuing with FIG. 1, the measuring head 116, attached by a sliding fixture 117 to base 101, is adjusted to position tension sensor 118 at a desired measurement point on a garment. With tension sensor 118 placed against the garment fabric, the tension of the garment at the measurement point can be read on digital display 119. Typically, an operator will take note of the tension reading, and use the data so obtained to calculate the corresponding pressure exerted by the garment at the measurement point. Further refinements in the HATRA measurement device not indicated on the drawings presented here, but familiar to any person skilled in this art, are mechanical aids to increase repeatability of measurements, and computer devices and software to record the digital tension data and perform the calculation of compression numbers using the Young-Laplace equation.

The HOSY measurement system consists of a computer unit (not shown) to automate the operation of the testing machine, perform calculations and create reports, and a measurement frame, shown in a schematic diagram in FIG. 3. The measurement frame has side rails 301, and an upper support beam 302. The garment to be tested is secured by stationary clips 304 on each end, and by multiple upper 305 and lower 306 testing clamps, arrayed in pairs. The upper testing clamps are supported by the header 302, and each lower clamp is attached to a vertical bar 307 which is in turn attached at its lower extremity to a stepper motor (not shown) which can move the vertical bar and attached clamp up and down under control of the computer. Each pair of testing clamps is 5 cm wide, and yields a fabric tension measurement for the 5 cm band of the garment to which it is attached. In FIG. 3, the system is shown with 14 testing clamp pairs in use. Up to 20 testing clamp pairs can be employed for a test.

In practice, a girth measurement for each testing zone is programmed into the computer system by an operator for a specific garment test. The stepper motor attached to each vertical bar will move, under control of the computer, increasing gap 308 between the each pair of upper and lower testing clamps to stretch the garment so as to tension the fabric to a degree equivalent to that which would be experienced if the garment were worn on a limb with the specified girth.

A more detailed schematic of a single pair of testing clamps is shown in FIGS. 4A and 4B. The upper support beam 401 and vertical bar 402 are shown. Two wire fixtures are placed inside the garment to be tested. The upper fixture 404 is a rigid rod, and the lower fixture 405 is a flexible coil, to allow independent movement of each lower clamp. The clamps are implemented as pairs of rollers, 406. The gap between each roller pair is adjusted to be small enough to trap the rod fixture, but wide enough to allow the fabric being tested to move through the clamp, equalizing tension on the two sides of the garment. FIG. 4B is a sectional view of the clamp rollers, showing the spacing of the rollers and the garment fabric. A load cell 407 in the upper clamp assembly measures the tension of the fabric at each clamp pair.

In a fashion similar to the HATRA system, the tension measurements from the HOSY system are converted to pressure numbers for each measurement zone using the Young-Laplace equation.

The HATRA and HOSY systems suffer from similar shortcomings. The accuracy of both systems is highly dependent on operator skill. Both systems were designed to measure garments with homogenous fabric structures, and are less accurate when measuring garments having asymmetrical side-to-side distribution of elastic fabric structures. In addition, the HATRA tester has difficulty measuring knit garments with patterned designs created with multiple yarns, as the device can produce inaccurate tension measurements at the points where the various pattern yarns stop and start in the pattern. The HATRA tester also is also limited to testing garments stretched to predetermined sizing profiles, and cannot easily be adapted to evaluate garments manufactured for the measurements of a specific, individual consumer.

Both the HATRA and HOSY devices, due to the fragility of the testing mechanisms and operational complexity, are unsuitable for use on the factory floor. As a result, evaluation and measurement of compression garments is performed well after manufacturing.

The third testing device for compression garments we will discuss, the medical stocking tester (MST) distributed by Swisslastic AG, is suited for some factory floor measuring needs. Shown in FIGS. 5A, 5B and 5C, the MST device provides measurement of garment pressure on pre-manufactured leg forms, for a small number of test points.

In FIG. 5A, the MST system is mounted to a pre-manufactured leg form 506. The form, typically constructed of wood or dense plastic, is manufactured to specific dimensions corresponding to a single, standard sock size. Each size being manufactured requires a separate leg form, and, ideally, a separate MST installation. The MST system consists of a flexible, transparent plastic envelope 501 in which are installed four sets of thin, paired conductive disk contacts 502 each attached to wire electrical leads 503. The electrical leads terminate in a manifold 504. The plastic envelope 501 is sealed to the manifold to provide an airtight enclosure for the contacts. The plastic envelope and disk contacts are adhered to the leg form 506 in such a way that the disk contacts are located at the marked girth points 507 to be measured. The manifold 504 is attached to a cable assembly providing both electrical power and pressurized air to the assembly, connected to a computerized test console (not shown.) FIG. 5C shows the disk contacts 502 in cross-section. It can be seen that the disk contacts 502 operate in pairs, with one adhered to the top and one to the bottom membrane of the plastic envelope 501. (For clarity, the dimensions of the plastic envelope, disk contacts, electrical leads and sock fabric in FIGS. 5B and 5C are not drawn to scale.)

In operation, a sock is placed on the leg form, mimicking the position in which it would be worn on a person's leg. The sock 508, not shown in FIG. 5A, can be seen in cross-sections FIG. 5B and 5C. The sock covers the plastic envelope containing the disk contacts. At the beginning of a test procedure, the envelope is deflated, and the disk contacts are touching, each pair completing an electrical circuit to the test console. For each pair of contacts in sequence, the console provides compressed air to the plastic envelope through the cable 505, increasing the pressure over several seconds. When the air pressure in the envelope exceeds the pressure of the sock fabric 508 on the leg surface, the upper and lower membranes of the plastic envelope are forced apart, shown in cross- section in FIG. 5C, separating the disk contacts 502, breaking the electrical circuit to the test console. The test console stops increasing the compressed air pressure, the final air pressure is noted, and the test console automation goes on to deflate the plastic envelope and test the next pair of contacts until pressure values have been determined for each contact pair.

While the MST tester provides a simple solution for some factory- floor compression garment testing, it has several shortcomings. First, it can only be used on fixed-size leg forms, and cannot easily be adapted for other sizes without manufacturing a new form. It can only measure a small number of points. The testing process is time-consuming, because of the need to test each measurement point in sequence. Finally, because of the small area of the plastic envelope and contact assembly, a patterned garment or a garment with non-homogeneous elastic structure which is not positioned over the plastic envelope will not be measured accurately.

In conclusion, each of the three commonly used systems, the HATRA, HOSY and MST devices, have shortcomings. The HATRA and HOSY devices do not measure garment pressure directly, but rather calculate pressure from tension measurements. While the MST system does take direct pressure measurements, it can only do so for a small, fixed number of points. While the HOSY system measures both sides of a garment, the HATRA and MST devices can produce inaccurate results when used on garments with asymmetric side-to-side distribution of elastic structure. The HATRA and MST devices will not yield accurate data if an area of a garment with interrupted threads, such as the cut yarn ends of a decorative pattern, coincide with the location of the measurement sensor. The HOSY device can be programmed to calculate pressure for arbitrary limb girth measurements, but both the HATRA and MST require the manufacturing of a new physical profile for a new series of girth sizes. Finally, the mechanical and operational complexity of the HATRA and HOSY devices make them difficult to use on the factory floor. Relying on measurements from either system, typically performed by an offsite testing laboratory, may entail delays of days or weeks between garment manufacturing and subsequent testing. Variability in manufacturing processes and raw materials, absent timely integration of measurement results into manufacturing quality control processes, can result in the manufacturing of compression garments differing substantially from the tested and certified sample products, to the detriment and confusion of consumers.

### SUMMARY OF THE INVENTION

This invention provides a measurement system usable in a testing laboratory and on the manufacturing floor for accurately modeling and measuring the pressure that will be applied to a limb by a compression garment. The system for accurately measuring compression garments in laboratory and manufacturing can also use the measurements to modify the programming of a manufacturing system to alter the compression parameters of subsequently manufactured garments. The system includes a support structure for insertion into a compression garment, and a plurality of sensor units at intervals along the support structure with each sensor unit extended circumferentially around the support structure to define a three-dimensional simulated anatomical form circumferentially stretching the compression garment upon insertion. Each sensor unit has a pressure sensor measuring the pressure exerted by the compression garment on the sensor unit after insertion.

Another object is to provide measurements of garment pressure for a variety of limb girths, including both standard industry garment sizes and customized measurements for an individual wearer based on the specific measurements of his or her body.

A further object is to provide a system of pressure measurement yielding accurate results for garments with asymmetrical elastic structures or with other non-homogenous yarn patterns.

Yet another object is to provide a measurement system for compression garments usable by relatively unskilled operators to produce accurate, repeatable measurements.

Still another object is to provide a means for automating the integration of compression pressure measurement data directly into manufacturing systems, to allow timely compensation for variations in manufacturing processes and raw materials.

These and other advantages, features, and objects of the present invention will be more readily understood in view of the following drawings and detailed description.

### BRIEF DESCRIPTION OF THE DRAWINGS

The present invention can be more readily understood in conjunction with the accompanying drawings, in which:

FIGS. 1 and 2 illustrate the HATRA device for measuring garment compression.

FIGS. 3 and 4A-4B illustrate the HOSY device for measuring garment compression.

FIGS. 5A, 5B and 5C illustrate the MST device for measuring garment compression.

FIG. 6A is a block diagram of the invention.

FIG. 6B illustrates the assembled invention on a stand, with a computer control tablet.

FIG. 7 presents an exploded view of the measurement array.

FIGS. 8A to 8G illustrate the construction of the sensor units of the measurement array.

FIGS. 9 and 10 document techniques for acquiring limb girth dimensions to use in setting up the measurement array.

FIGS. 11 and 12 are flowcharts presenting the operation of the invention.

FIGS. 13A to 13D illustrate the control application interface on the screen of the control tablet.

FIG. 14 illustrates the measurement array with a sock in place to be measured.

FIG. 15 provides an example of the data and calculations required to change the settings of a knitting system to adjust the compression of a knitted garment.

FIG. 16 presents a flow chart detailing the process for modifying knitting system compression parameters.

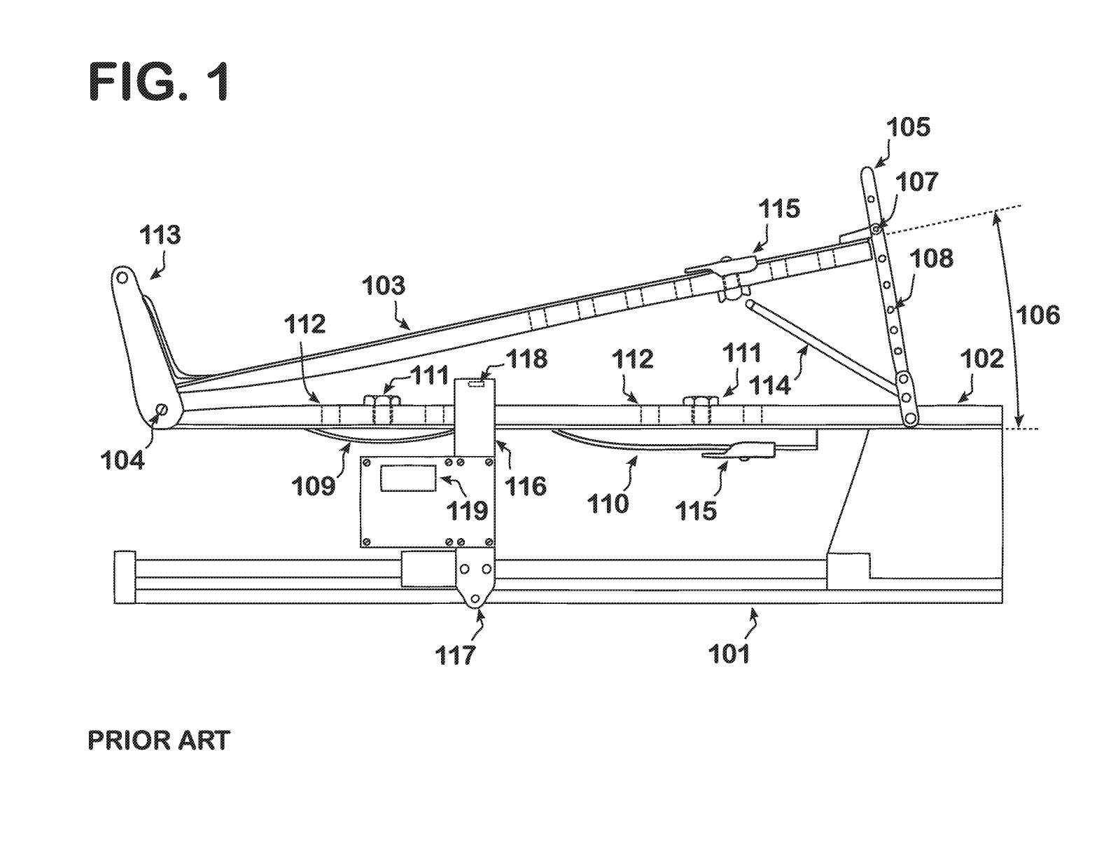

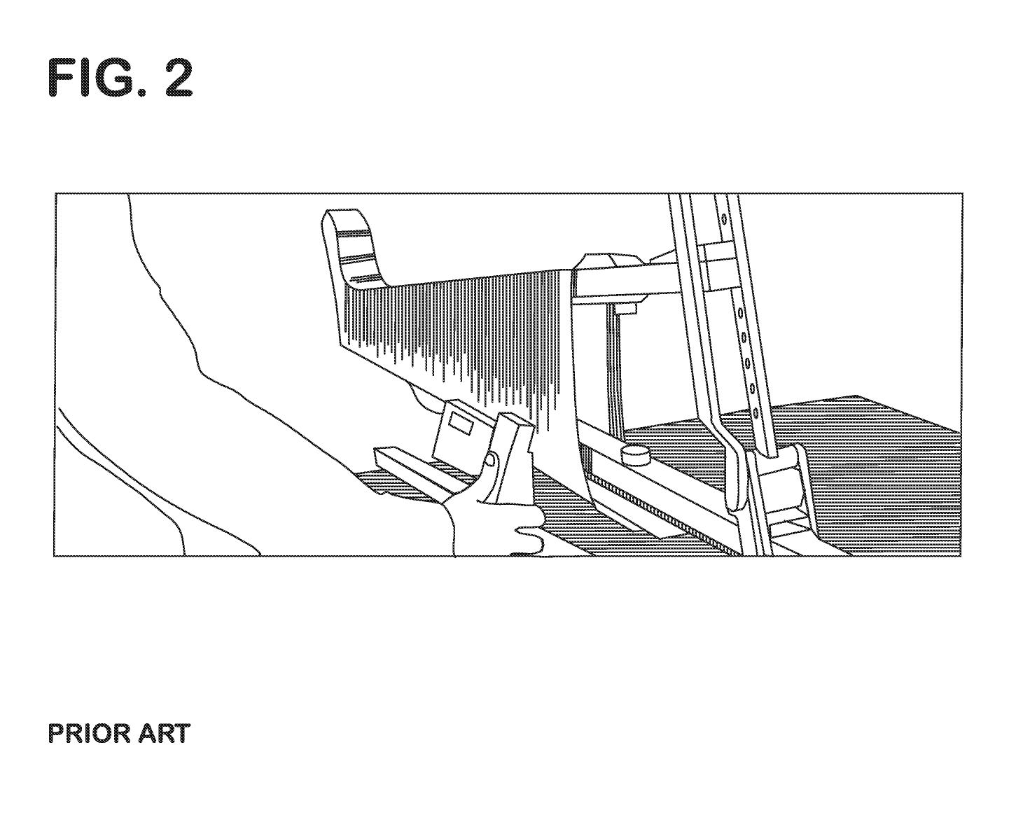

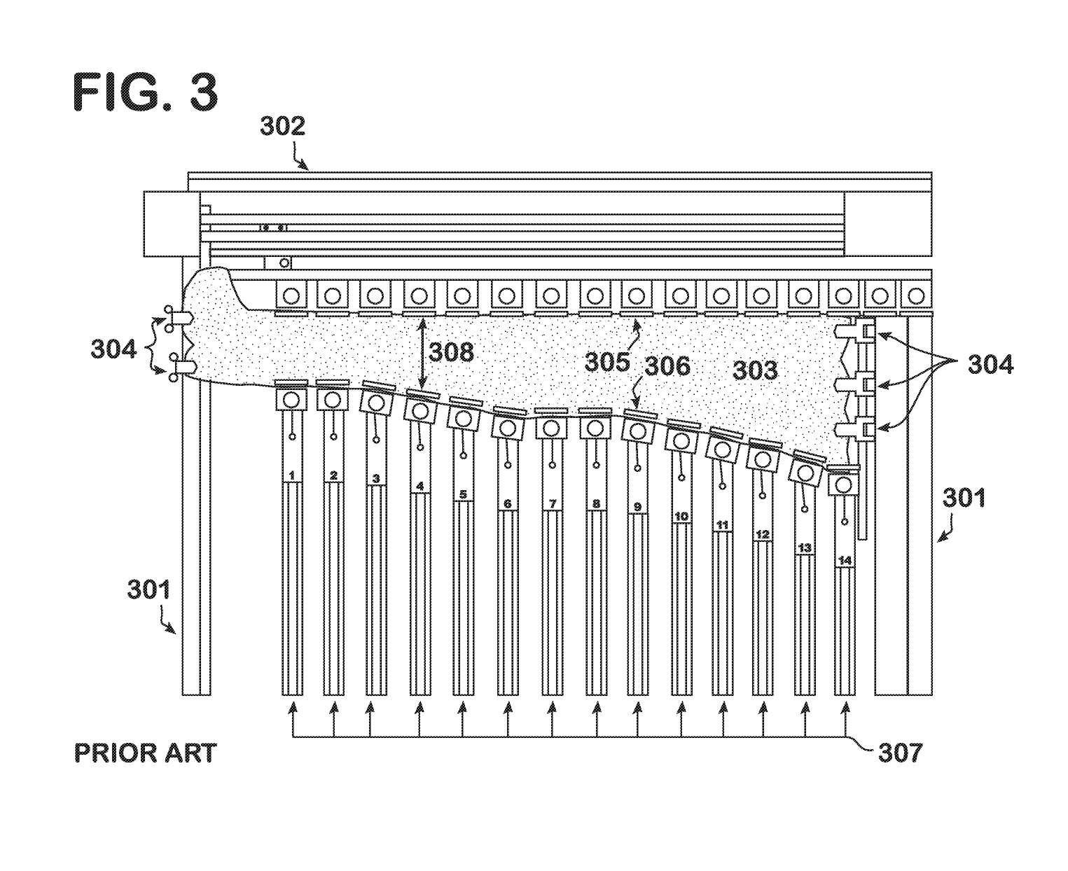

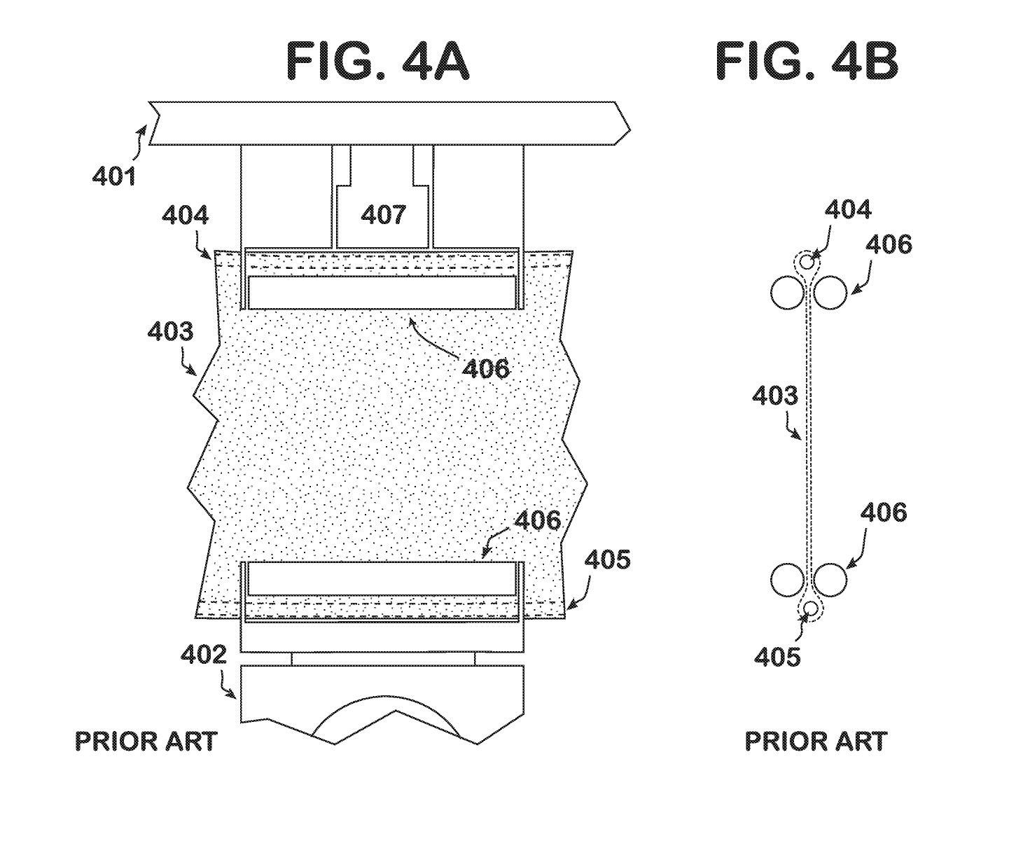

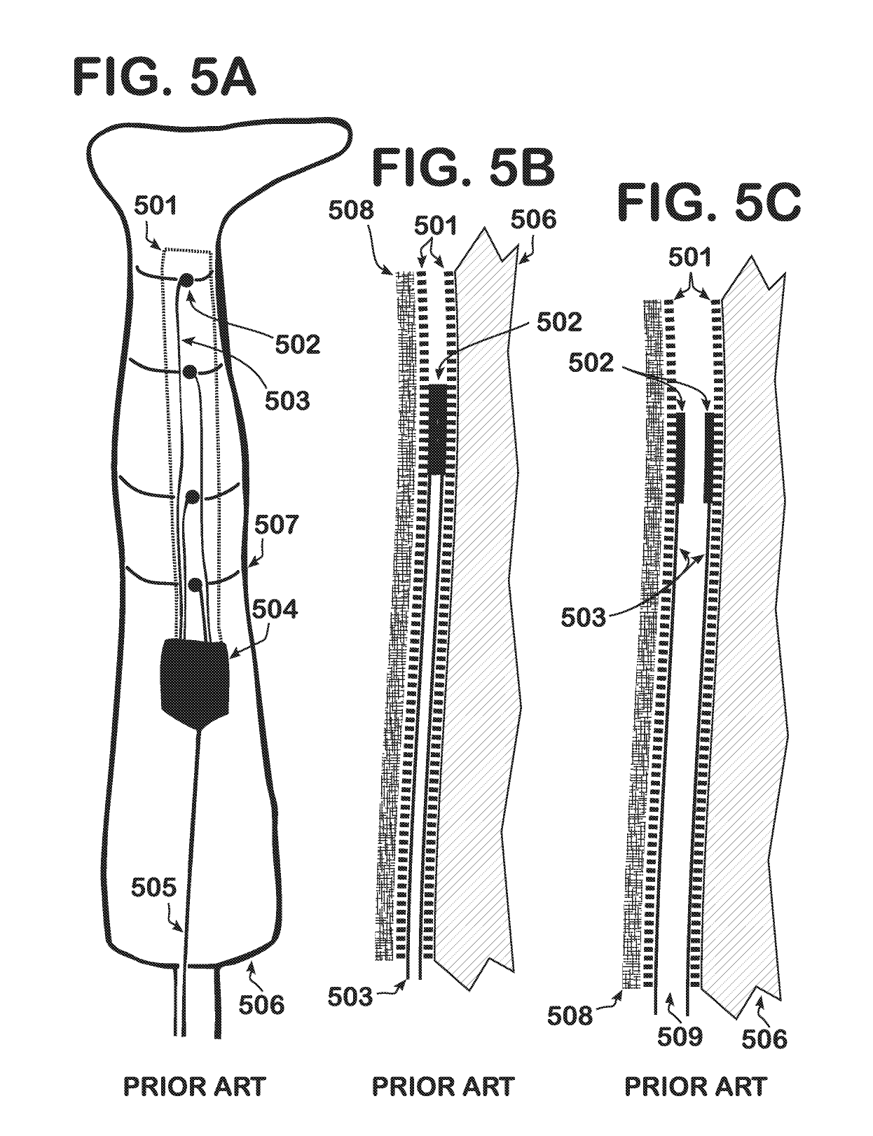

### DETAILED DESCRIPTION OF THE INVENTION

In general terms, the present invention provides a system for accurate measurement of compression garments. The invention measures pressure directly at multiple regions of a garment, and measures the totality of pressure the garment will apply around the entire circumference of a limb, rather than inferring the pressure from a tension measurement. Because of this, it can be used to measure the pressure exerted by garments with asymmetrical elastic structures, or with extensive colored yarn patterns having many cut ends. Such garments could yield inaccurate results from single-point tension measurement systems, such as the HATRA or MST devices. The current invention can be used in both laboratory and manufacturing venues, and therefore provide timely information during manufacturing operations. Having measured a garment from a production knitting system, the invention can generate compensating program data for subsequent manufacturing runs, to correct any irregularities in the initial compression profile.

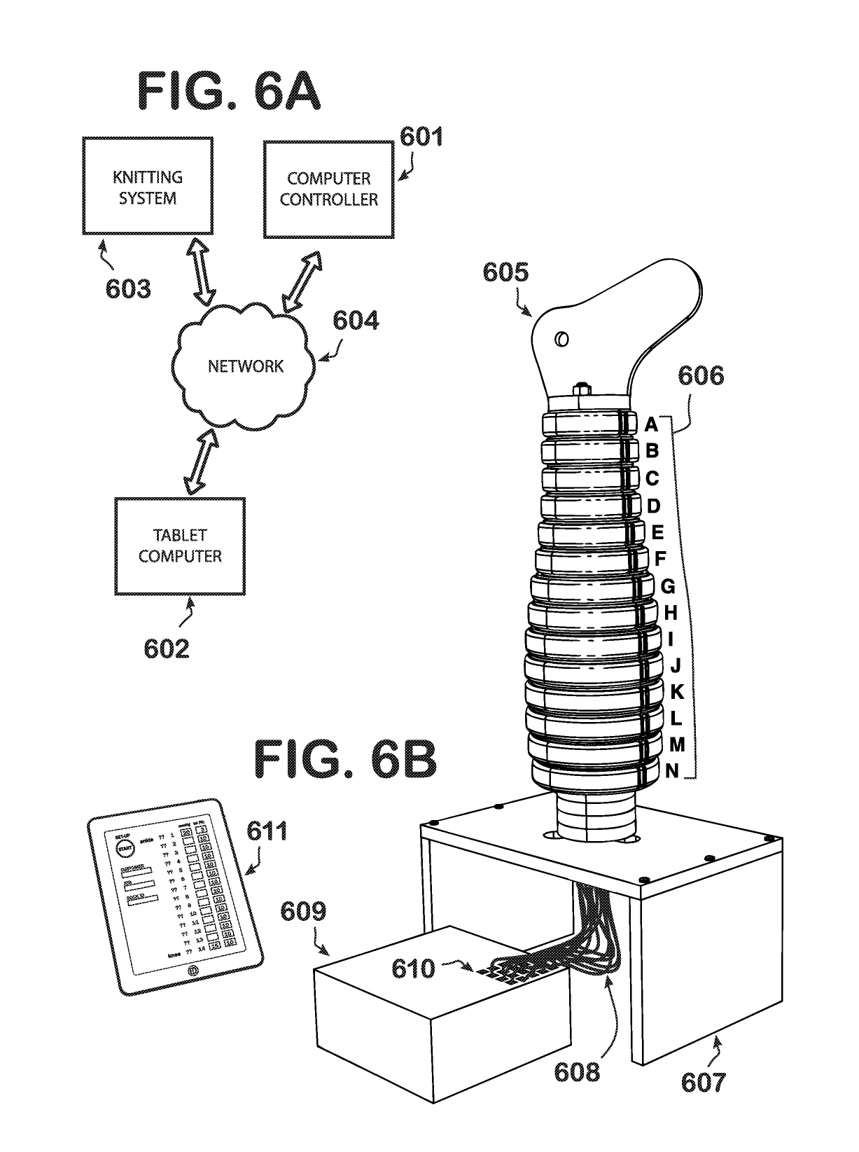

A block diagram of the invention is shown in FIG. 6A, including a computer controller 601, a commercially-available tablet computer 602 employed to run graphical user interface control software, and a knitting system 603 are connected to a computer network 604 by wired or wireless connections. FIG. 6B shows an embodiment of the invention having a measurement array 605 over which a garment can be placed to be measured. The measurement array 605 consists of multiple sensor disks 606, here labeled A to N. The measurement array 605 is mounted on a stand 607, and is connected by multiple electrical cables 608 to a computer controller 609. The computer controller has a multiplicity of electrical jacks 610, into which the electrical cables are plugged. A commercially-available tablet computer 611 running a customized software application is employed to set up and control the measurement system.

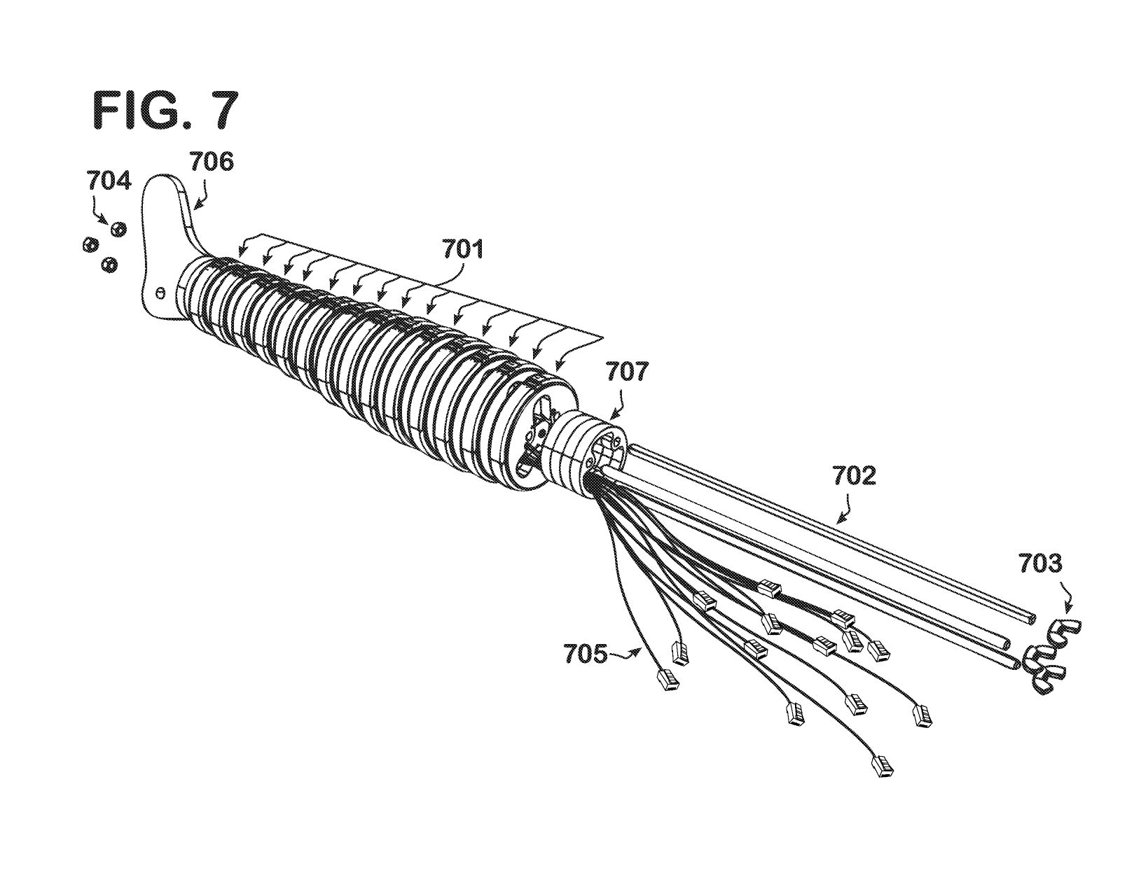

FIG. 7 presents an exploded view of the measurement array. The array consists of multiple sensor units 701 assembled on three alignment rods 702 that serve as a support structure for the sensor units 701. The alignment rods and sensor units are secured on each end with nuts 703 and 704. It should be noted that other types of support structures could be employed to interconnect and support the sensor units 701. For the purposes of this disclosure, the term “support structure” should be broadly construed to include any structure, mechanism, connectors or brackets used to interconnect and support the sensor units so that the assembly of the support structure and sensor units defines a desired three-dimensional simulated anatomical form (e.g., a leg or foot) for insertion into a compression garment.

Each sensor unit has a wired electrical connection, and the wires 705 are threaded through the center of each successive sensor unit, exiting at one end of the measurement array. For hosiery, there is a foot piece 706 to help support the foot of a sock, and series of spacer rings 707 are added to allow fabric longer than the array to extend below the last sensor unit so as not to interfere with the operation of the sensor unit. It should be noted we have chosen to use circular sensor units, arranged in a concentric stack. While a person's anatomy is neither circular nor symmetrical in cross-section, the pressure measurements produced by the invention will accurately predict the pressure a measured garment will exert on a person's limb if the circumference of the sensor units in the measurement array are matched to the girth measurements of a limb.

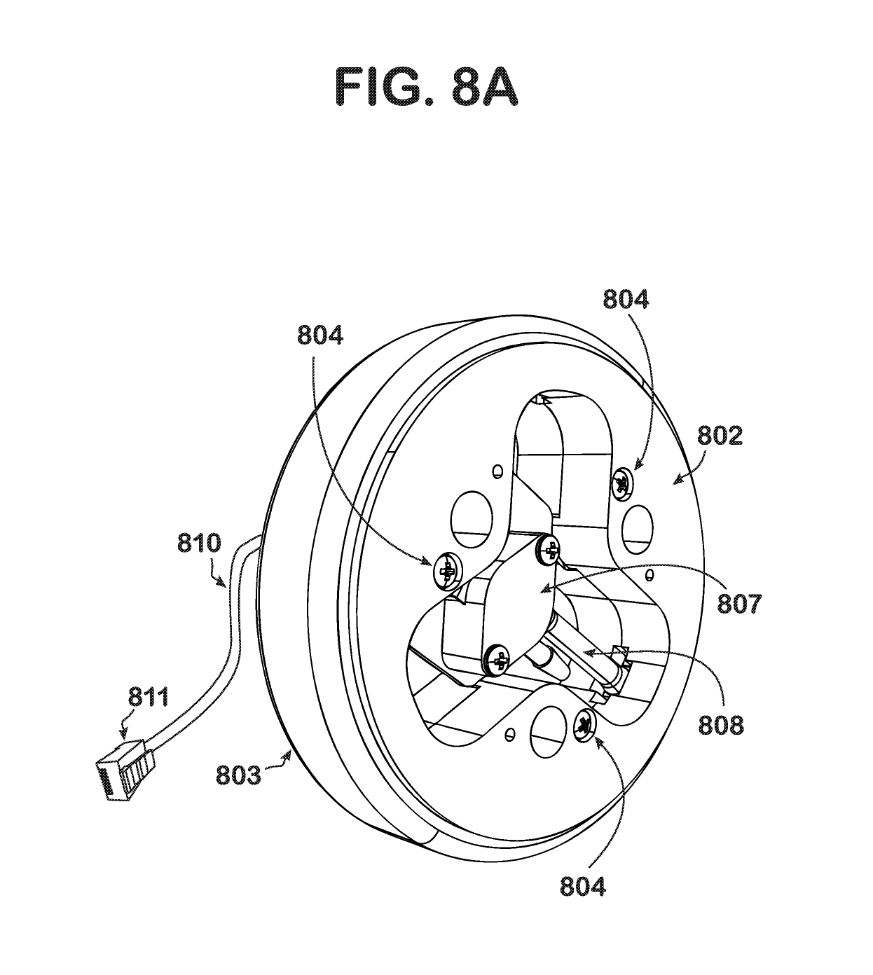

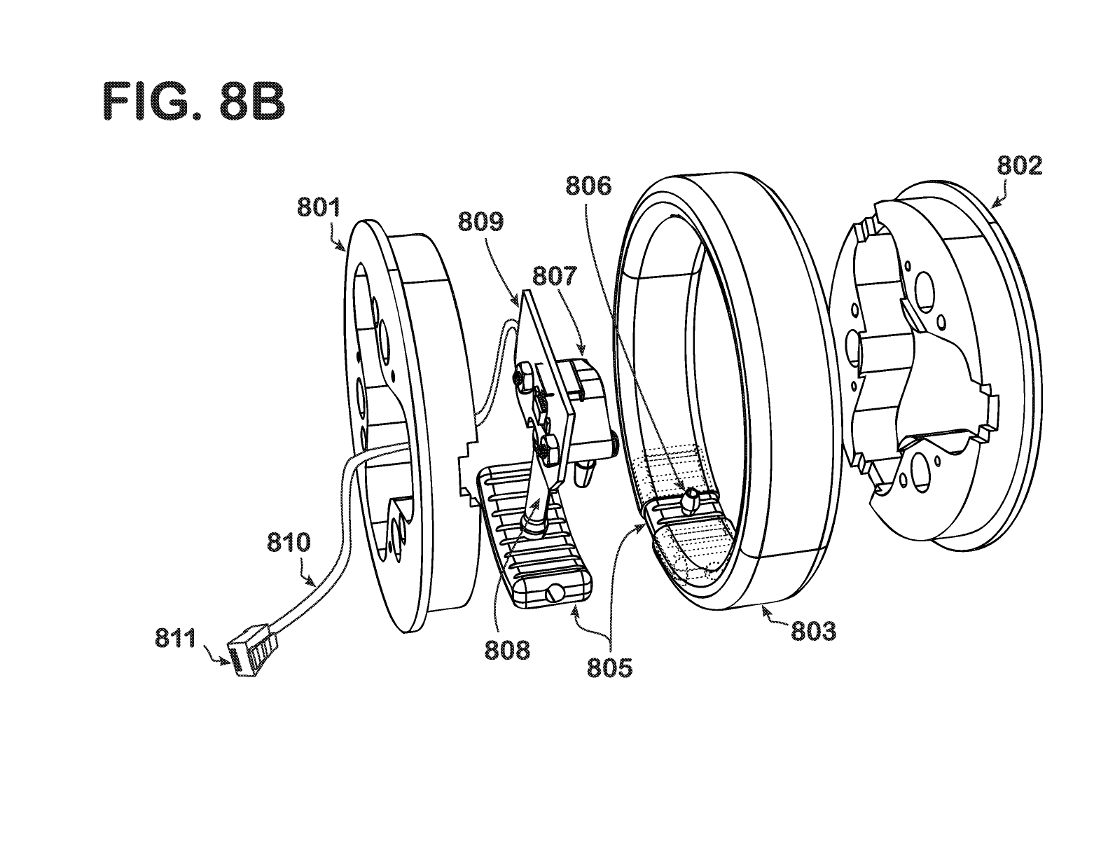

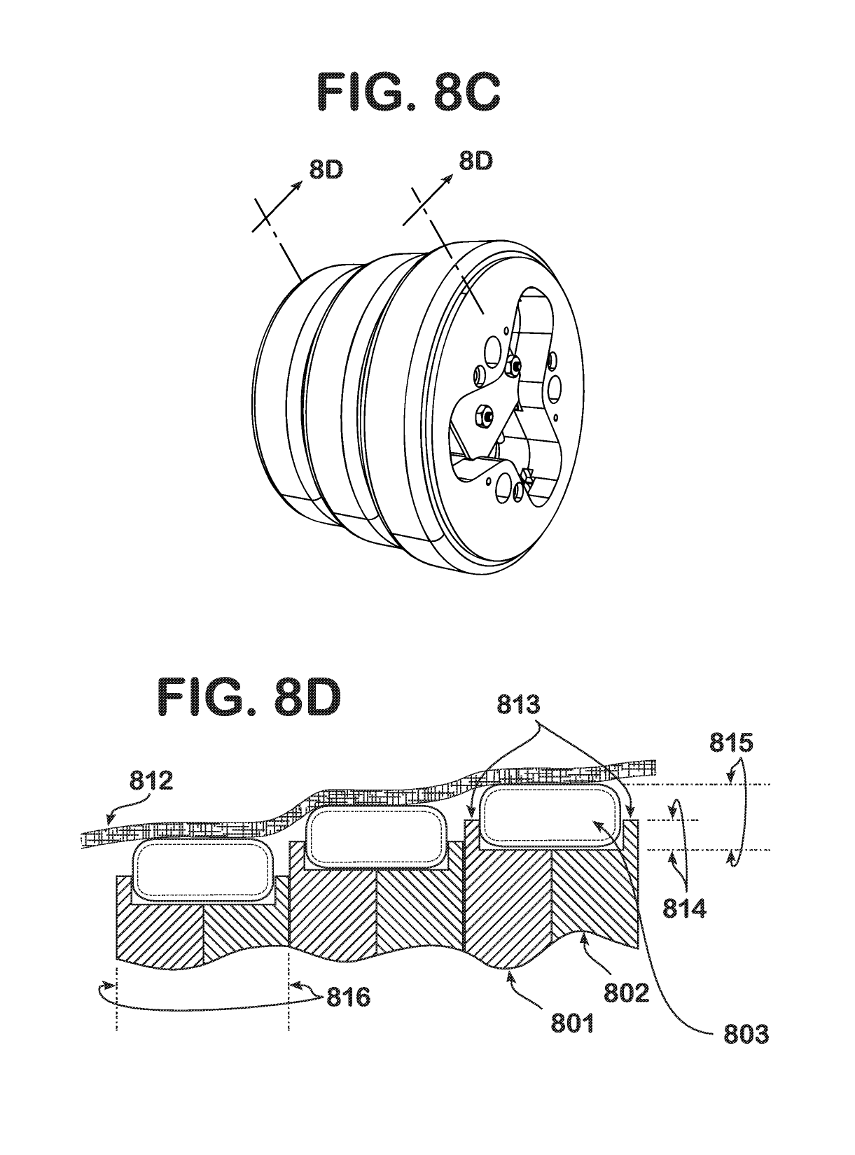

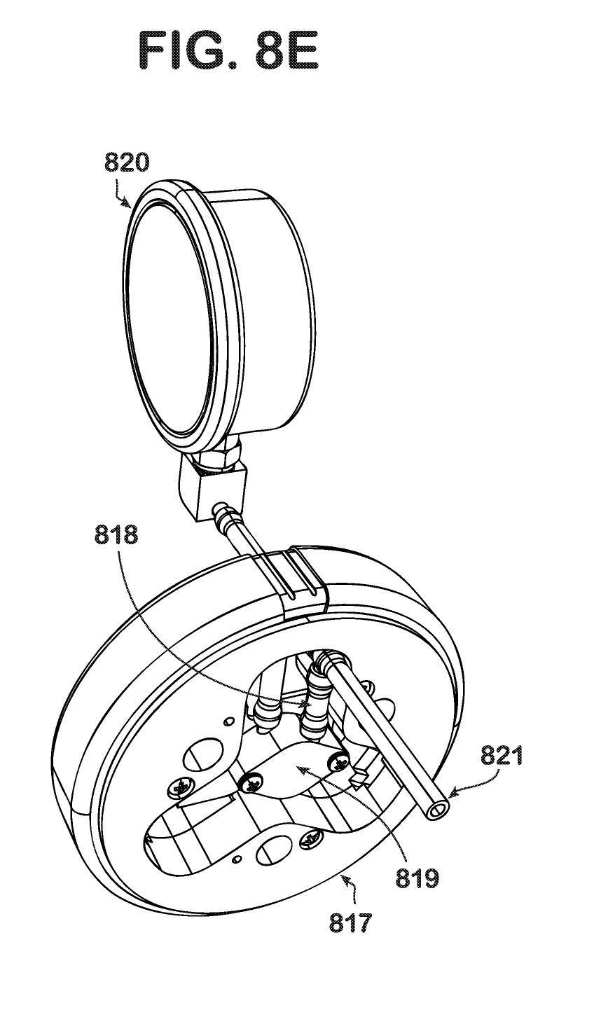

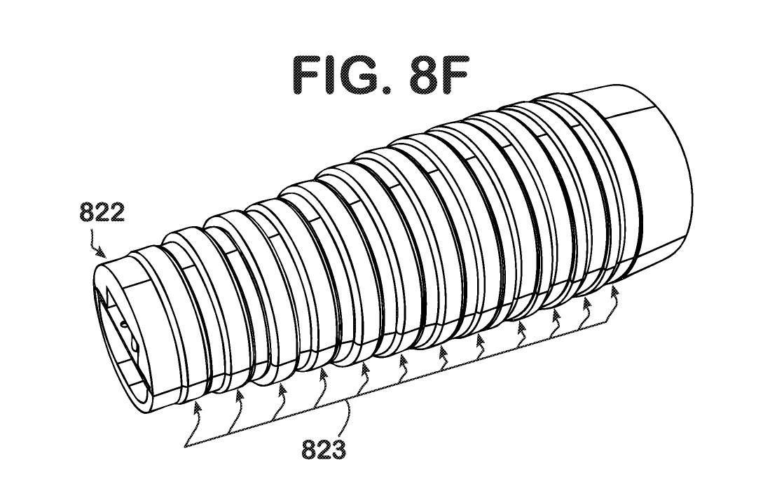

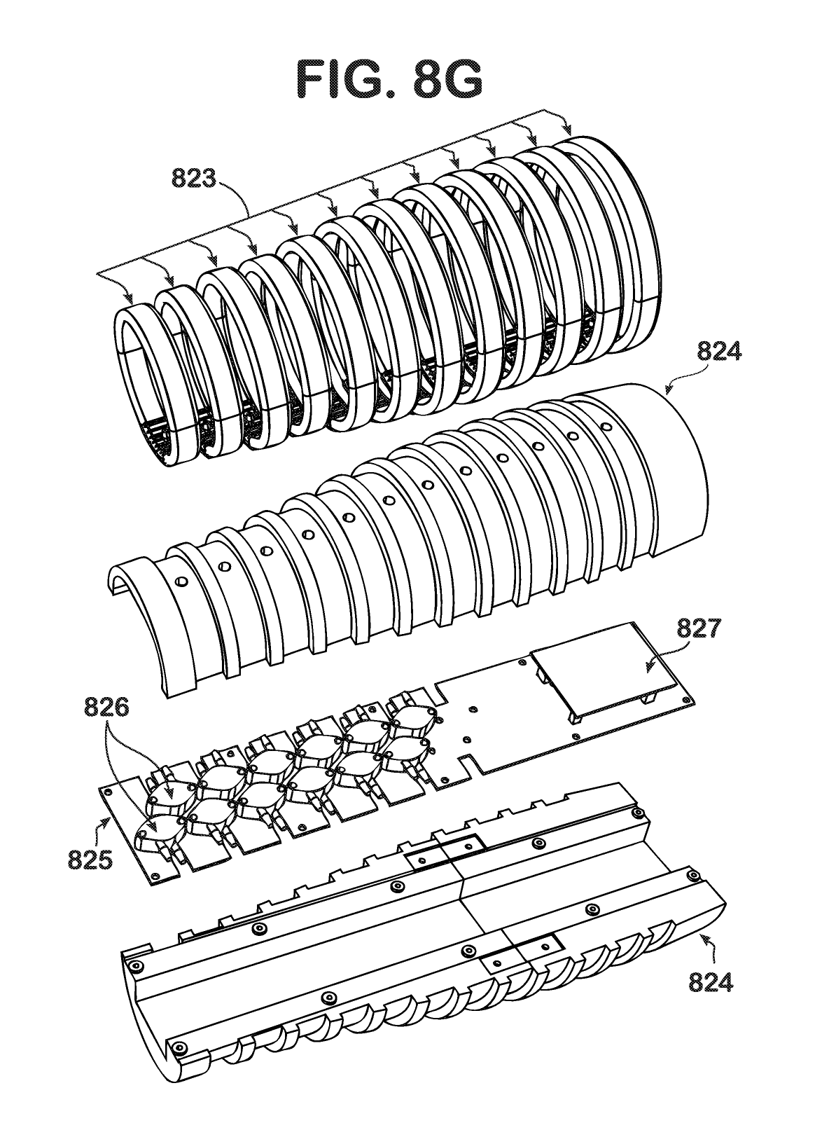

An individual sensor unit is shown in FIG. 8A, with an exploded view of its constituent parts in FIG. 8B. Each unit consists of two metal or plastic supporting disks, 801 and 802, supporting a hollow, flexible tube 803, which, in varied embodiments, can be filled with either air or a fluid. The two machined disks are secured to one another by screws 804. The machined disks and flexible tube are sized such that the outer circumference of each tube 803 is equal to the girth of the limb for which garment pressure is to be measured at a specific point on the garment. The ends of the flexible tube 803 are joined by a curved, hollow, ridged coupling 805. At the center of the coupling is a tubing barb 806, to which is connected an electronic pressure sensor 807 by means of a tube 808. The assembly of the flexible tube, coupling, and pressure sensor must be free from leaks, as any escaping air or fluid will impair the accuracy of the pressure sensor. Sensor 807 is mounted on an electrical circuit board, 809. Omitted from this drawing, but which will be readily understood by those skilled in this art, is a port and tube assembly for filling and pressurizing the flexible tube 803.

Those skilled in the art will recognize that the subsystem consisting of the flexible tube 803, ridged coupling 805, connecting tube 808 and electronic pressure sensor 807 can be replaced, in alternate embodiments, with other pressure sensing technologies capable of electrically reporting the circumferential pressure applied by a garment to the circular sensor unit. Such technologies include but are not limited to electronic force-sensitive resistors, piezoresistive sensors and piezocapacitive sensors.

Finally, there is a multi-conductor electrical cable 810 attached to the electrical circuit board with a terminating plug 811. The electrical cable provides power for the pressure sensor, and is also used to pass configuration, calibration and measurement data between the pressure sensor and the computer controller 604 shown in FIG. 6.

In FIG. 8C, three sensor units are shown in an interconnected stack, to give context for the cross-section in FIG. 8D. The flexible tube 803 is supported in each sensor unit by supporting disks 801 and 802. The fabric of a garment in cross-section 812, not shown in FIG. 8C, has been added to illustrate the relationship between the fabric being measured, the flexible tubes and the supporting disks. The raised edges of the supporting disks 813 limit the side-to-side motion of the flexible tube. The height of the raised edges of the supporting disks 814 must be less than the height of the flexible tube 815 when the tube is filled with air or fluid, to avoid interfering with the compressive action of the fabric upon the flexible tube during the measurement process.

The width of each sensor unit 816 in the current embodiment is one inch, providing a compression measurement corresponding to a 1-inch wide segment of the garment placed on the device. However, it will be readily apparent to those skilled in this art that sensor units may be constructed in various widths, wider or narrower, based on the desired measurement resolution.

In FIG. 8E, we show a sensor unit 817 with tubing 818 attached to the back port of the electronic pressure sensor 819. The back port is unused in measurement operation, and is normally open to ambient atmospheric pressure. By placing a calibrated pressure gauge 820 on the tubing assembly and using a hand pump or other means (not shown) attached to the end of the tubing 821 to evacuate the pressure in the tubing to a moderate vacuum, a pressure measurement may be obtained for a span calibration of the electronic pressure sensor. Span calibration may be necessary at the initial assembly of the sensor unit, and occasionally as dictated by any change in the operating characteristics of the sensor over time.

If the ability to modify the measurement array for various girth measurements is not required, an alternate embodiment of the invention 822 is shown in FIGS. 8F and 8G. In this embodiment, instead of separate disks being used to support the flexible tubes 823, a plastic or metal form, comprised of two halves 824, is used as the support structure to support multiple sensor units. Here again, each sensor unit includes a flexible fluid-filled tube and a pressure sensor. (The tubing connections between the pressure sensors and the flexible tubes are not shown.) Unlike the stacked disk measurement array in FIGS. 6 and 7, the form will not be frequently disassembled and reassembled. As a result, the interior space can be used as a housing for the electronics of the computer controller. In FIG. 8G, we show flexible tubes 823 and a circuit board 825 with multiple electronic pressure sensors 826 mounted to the circuit board. It is possible to include the pressure sensors, the required sensor support electronics and the control processor 827 in the same interior space.

Returning to FIG. 8A, we note the pressure exerted on a limb by a given compression garment is determined in part by the girth of the limb being measured. The circumference of the flexible tube 803 and supporting disks 801 and 802 in a specific measuring scenario are chosen to match the circumference of the limb or body part for which measurements are being taken. While the following discussion will focus on the measurement of pressure for compression hosiery, those skilled in this art will recognize that the sensor units in alternate embodiments can be sized so as to provide appropriate circumferences for a leg, arm, torso, pelvis or other portion of a person's anatomy.

The measured circumference of a person's limb will vary based on many factors, including the position of the limb, the state of activity of the underlying musculature, and the duration and energy of recent exertion. Creating a series of sensor units in circumference increments of one centimeter allows us to approximate real-world girth measurements closely enough for accuracy, and also places a realistic bound on the number of sensor units needed to test a reasonable range of girths.

It is a common practice in the hosiery industry to manufacture garments to standard sizes agreed upon by trade or government associations. For instance, one such association in the United States was the National Association of Hosiery Manufacturers, NAHM. (NAHM as an association is now defunct, but the organization's intellectual property survives and continues to be managed by the Manufacturing Solutions Center in Conover, N.C.) To support the creation of more uniform sizing for consumers, NAHM produced sizing boards to be used in the evaluation of hosiery. The dimensions of the boards are derived from the averages of girth measurements taken from a large number of consumers.

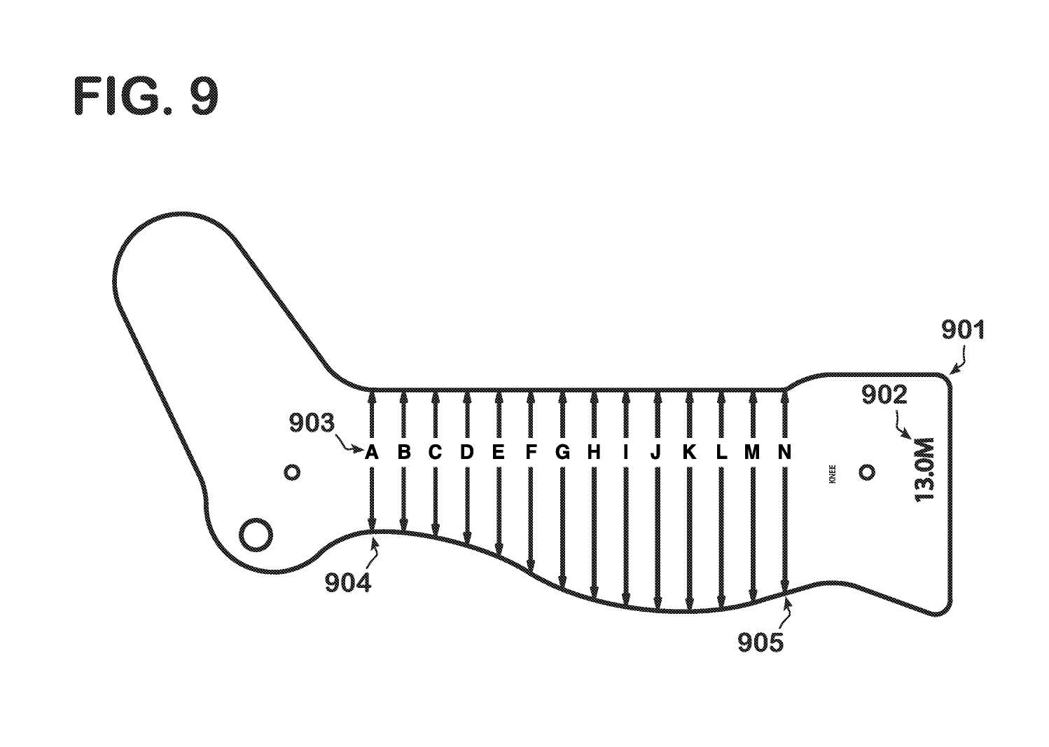

One such board 901 is shown in FIG. 9. The circumference of the board at a measured point is equivalent to the average girth, as determined by NAHM, of a leg for a person who would wear the documented size. Shown is a board labeled 902 for a men's size 13 sock. To set up the measurement array for use, one approach is to take the measurements from such a board as an approximation of the desired garment size. For the embodiment of the current invention having a sensor unit width of one inch, measurements can be taken at 14 one-inch increments, shown by labels A to N. Position A 904 provides the ankle girth measurement, and position N 905 the measurement just below the knee. Having taken the series of measurements, each measurement is rounded to the nearest centimeter, and the appropriate sensor units with the derived circumferences are assembled in a stack, as shown in FIG. 7.

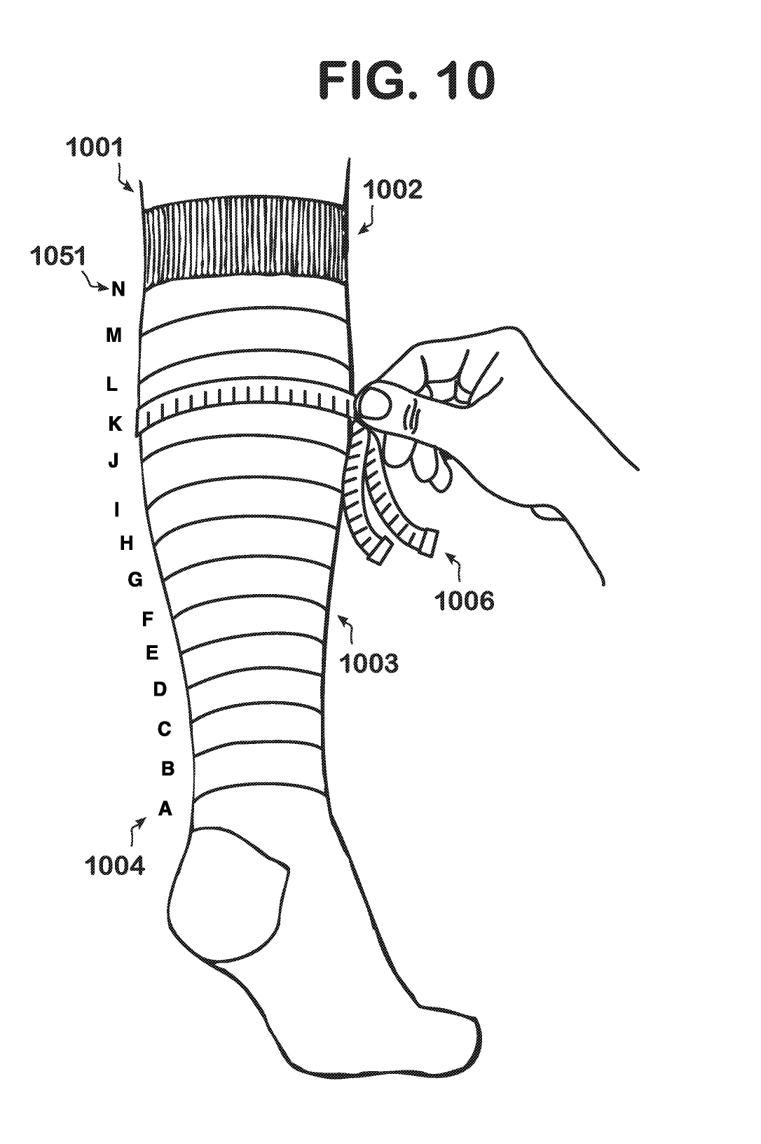

An alternative method for choosing the sensor units for the measurement array is to measure a specific person's limb. Shown in FIG. 10 is a person's leg 1001. The person has donned a sock 1002 with markings 1003 denoting the circumference every one inch. The markings may be drawn in ink upon a plain sock, or may be knitted into a sock manufactured for this purpose. In similar fashion to the NAHM board illustrated in FIG. 9, label A 1004 is at the ankle, and label N just below the knee. Using a suitable measuring tape 1006, a set of girths may be measured, rounded to the nearest centimeter, and those derived measurements employed to choose appropriate sensor units. The compression data gathered by the invention for such a series of personal measurements will accurately model the compression performance of a garment when subsequently worn by the specific individual.

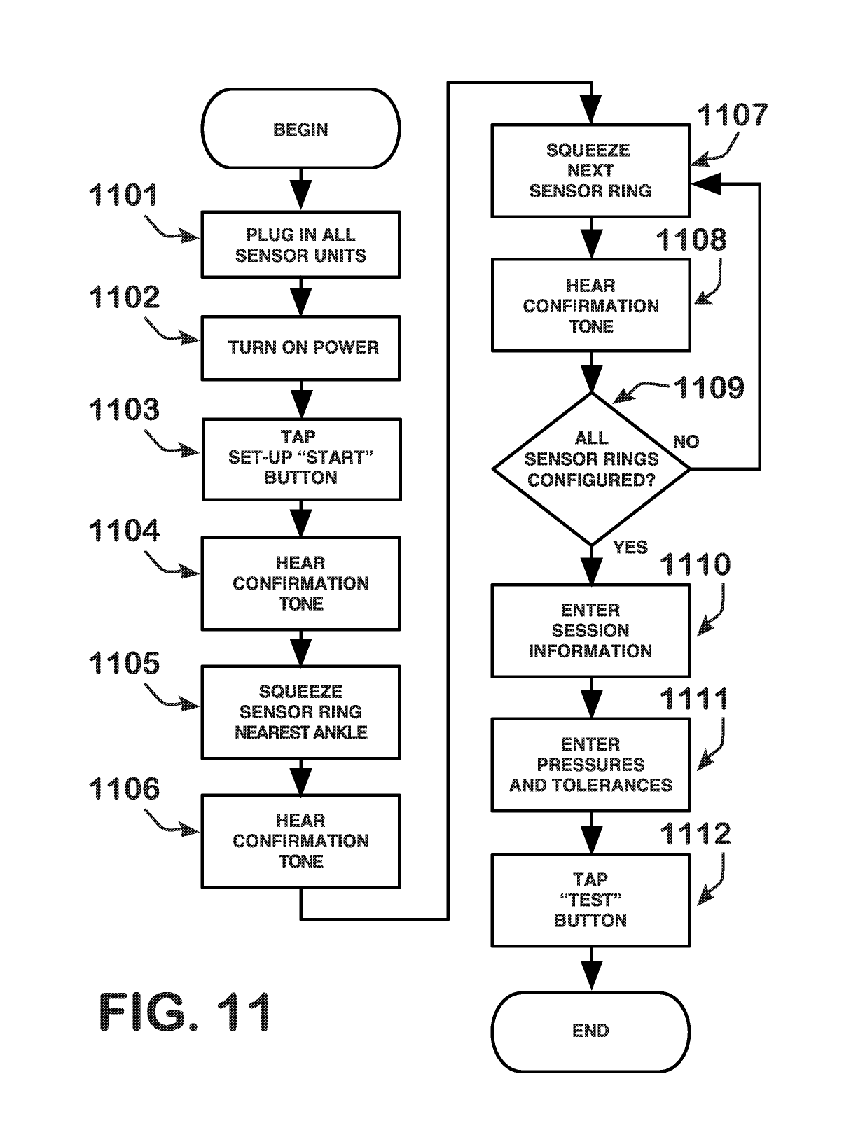

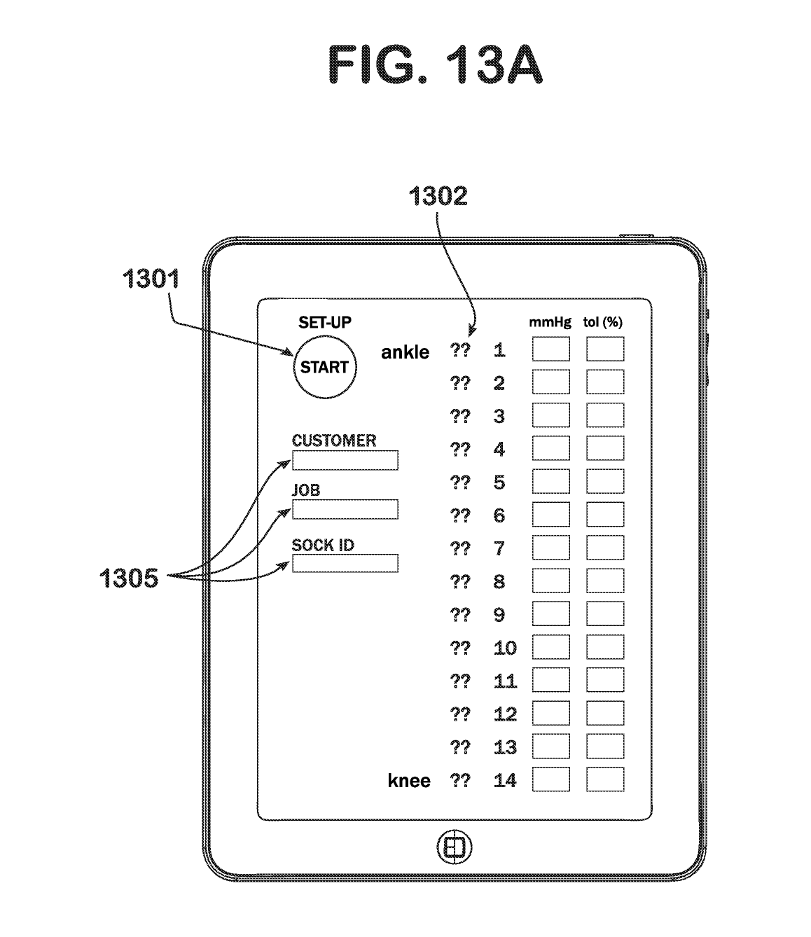

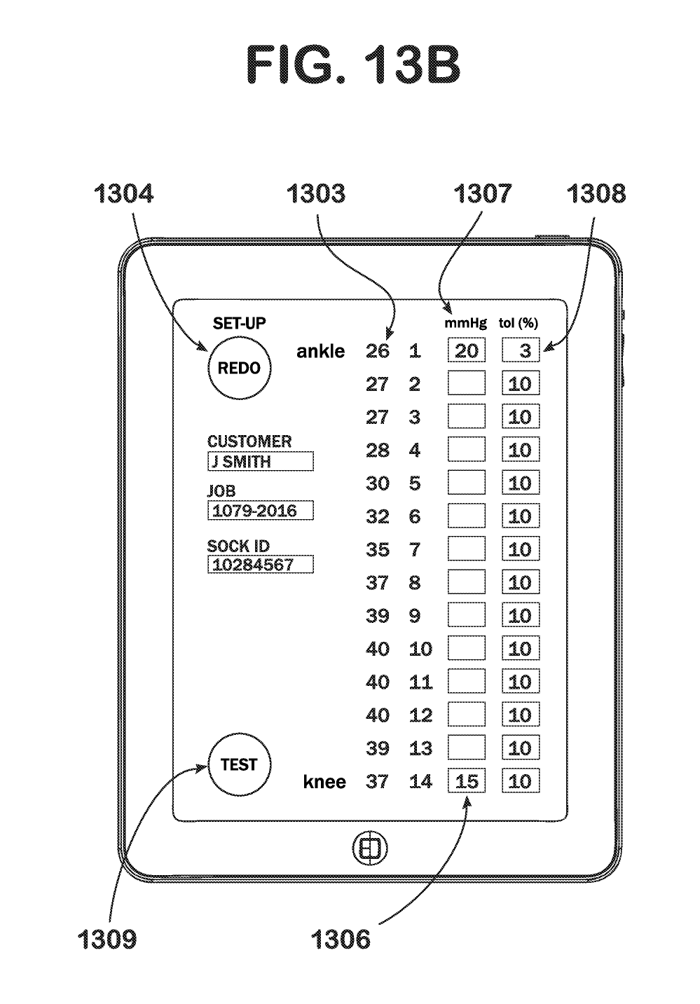

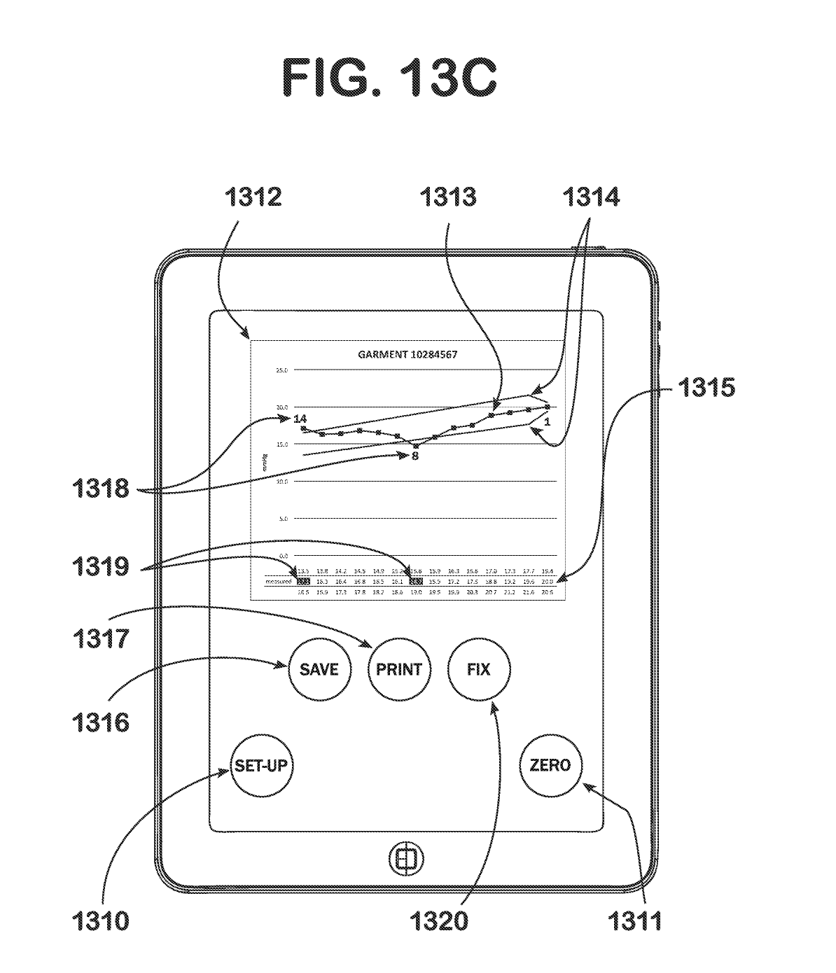

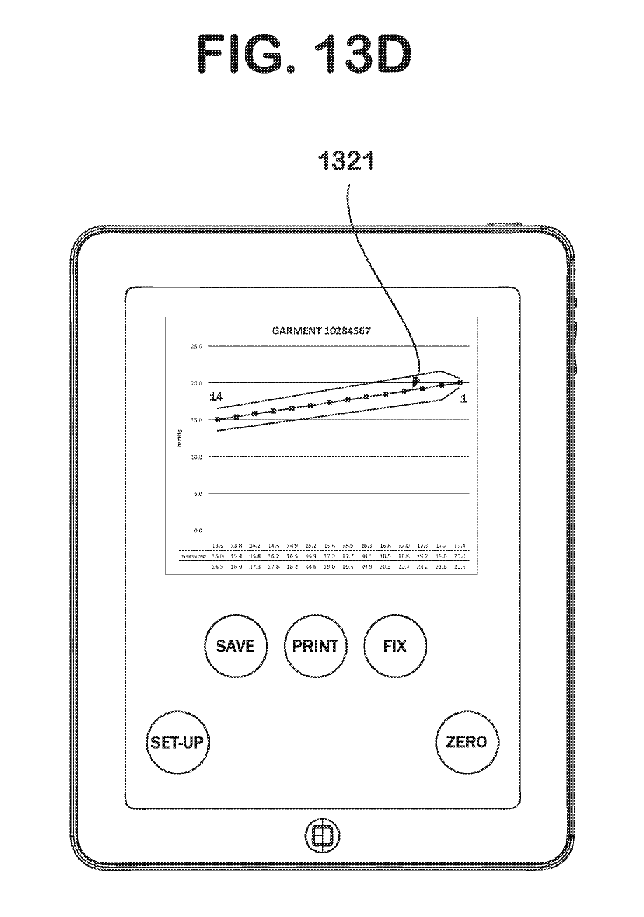

To employ the invention to measure the pressure exerted by a garment, we follow the process documented in the flow chart shown in FIG. 11, and observe the interface for the control application illustrated in FIGS. 13A-13D. To begin, all sensor units must be plugged in 1101 to the sensor jacks on the computer controller unit. (604 in FIG. 6.) Any sensor unit cable may by plugged in to any sensor jack. There is no required order or sequence, as the arrangement and identity of the individual sensor units will be determined during the set-up process. The computer controller and tablet computer are powered on 1102. The operator then turns his or her attention to the control application on the tablet computer (606 in FIG. 6) It should be noted the visual interface presented by the control application on the table computer is shown in a simplified, schematic form. Various visual details, prompts and user interface affordances have been omitted for clarity.

The initial display on the tablet computer is shown in FIG. 13A. The operator initiates the set-up process 1103 by tapping the “START” button on the tablet screen 1301. The tablet will produce an audible confirmation tone 1104. The operator then gently squeezes the flexible tube of the sensor unit nearest the “ankle” of the measurement array with his or her fingers 1105 to identify the sensor ring to the computer controller. The tablet will produce an audible tone to confirm the computer controller has registered the increase in pressure in the sensor unit, has read the identifying serial number and circumference information from the sensor unit electronics, and has configured the sensor unit to be the first sensor unit in the measurement array 1106. The operator will then squeeze the flexible tube of the next sensor unit in sequence 1107, hear the confirmation tone 1108, and proceed through the remainder of the sensor units until all sensor units have been configured 1109. As the operator proceeds through identifying each sensor unit to the computer controller, the “??” annotation on the tablet display 1302 will be replaced by the circumference measurement of the sensor unit 1303. Upon completion of the sensor disk set-up process, the label of the “START” button 1301 will change to “REDO,” 1304. Tapping the “REDO” button at any time after this point in the process will reinitiate the sensor disk set-up process, beginning at step 1104.

The operator will now fill in any identifying session information for the measurement test to be performed 1110. Areas to fill in customer, job and sock identifiers are shown 1305, but it will be readily apparent to anyone skilled in this art that the information required can be modified to suit the particular production process for a specific embodiment. In addition, the operator will specify the compression targets for the garment to be tested 1111. If at least a starting 1306 and ending 1307 pressure are provided and any target pressures remain unsupplied, the application will automatically calculate the intermediate pressure target values. The operator will also supply the tolerances in percentage values for the system to use in evaluating the compression garment at each sensor unit 1308. When all required values have been entered, the display will be similar to FIG. 13B, and the “TEST” button 1309 will be active on the display. When the operator has completed entering values to his or her satisfaction, they may proceed to tap the “TEST” button on the computer tablet display, completing the set-up process 1112. The computer tablet display will change to the view shown in FIG. 13C. At any time, the operator may tap the “SET-UP” button 1310, and return to the set-up display of FIG. 13B to change the set-up parameters.

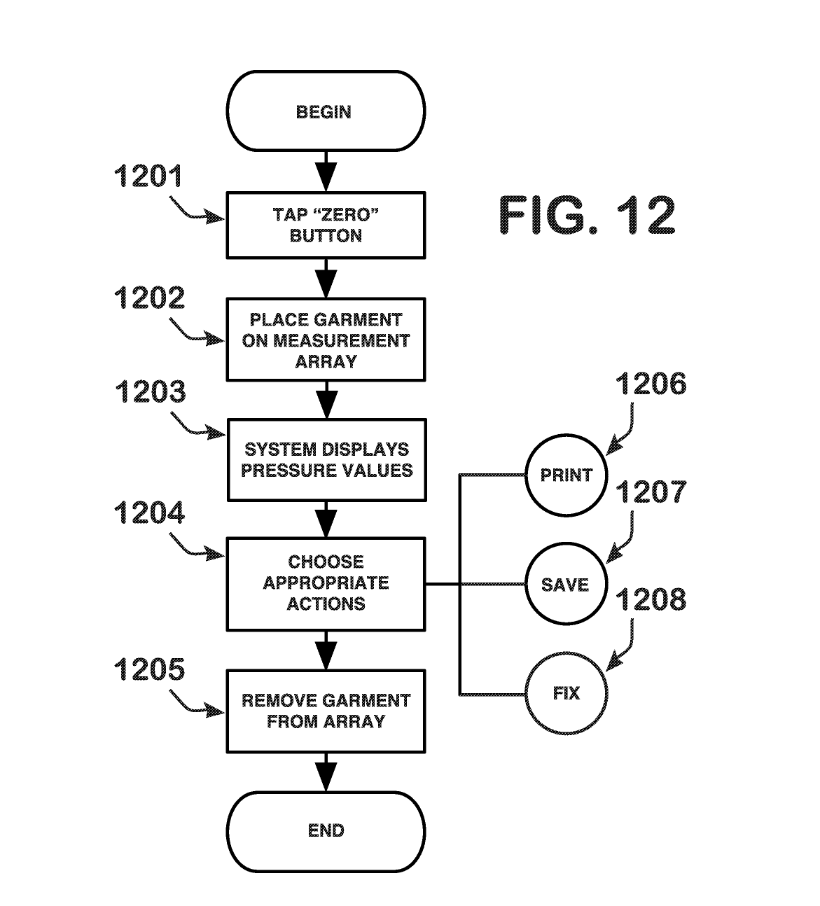

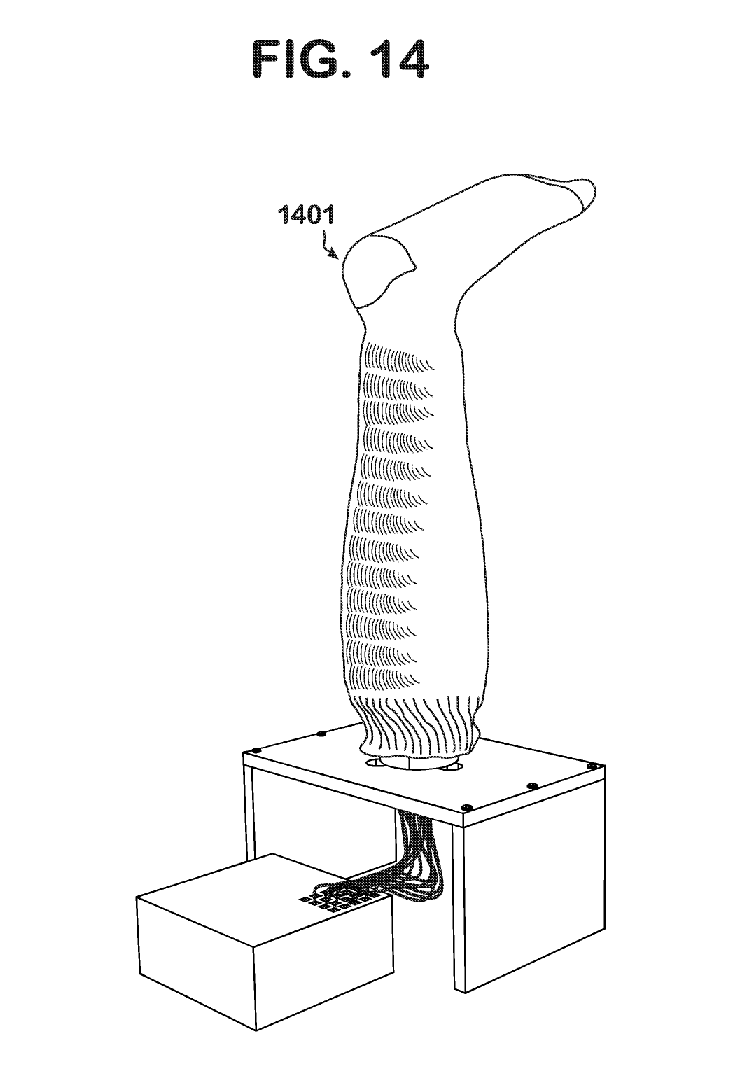

FIG. 12 presents a flow chart summarizing the process of using the system to measure the pressure exerted by a garment. After completing set-up and before placing a garment on the measurement array, the operator may zero the system 1201 by tapping the “ZERO” button 1311 on the computer tablet display. The system will then read the pressure reported by each sensor unit, and store the value as the relative “zero” value for the sensor unit. Zeroing the system before each garment is measured is desirable, as the system will be affected by short-term changes in ambient atmospheric pressure; the zeroing process compensates for those changes. To measure a garment 1202, the garment 1401 is placed on the measurement array as shown in FIG. 14. Returning to the computer tablet display, shown in FIG. 13C, the operator will see the system will continuously report the pressure 1203, relative to the zero value, exerted by the garment on each sensor unit. The pressure values from the sensor units will be reported in a data display area 1312 on the tablet display as a series of data points 1313 in a graphic chart. The tolerance range for each sensor unit, as entered in the set-up operation, will be displayed as lines 1314 above and below the data points. In addition, a table 1315 of the numeric values for the measured pressures and tolerance values is displayed. At this point in the measurement process 1204, the operator may tap the “SAVE” button 1316 to store the results to a computer disk or database, or tap the “PRINT” button 1317 to send the results to a printer.

If any measured sensor disk pressures are outside the tolerance range specified by the operator, those data points will display outside the tolerance lines 1314 on the display and the data points will be labeled with an identifier for the specific sensor disk providing each pressure measurement 1318. In addition, the numerical data for the out-of-tolerance data measurements will be highlighted 1319 in the table of numeric values 1315.

The system also provides a means to modify the compression data used by the knitting system that created the garment being measured, in order to adjust the compression profile of subsequent manufactured garments. If desired by the operator, he or she may tap the “FIX” button 1320 to initiate this process,

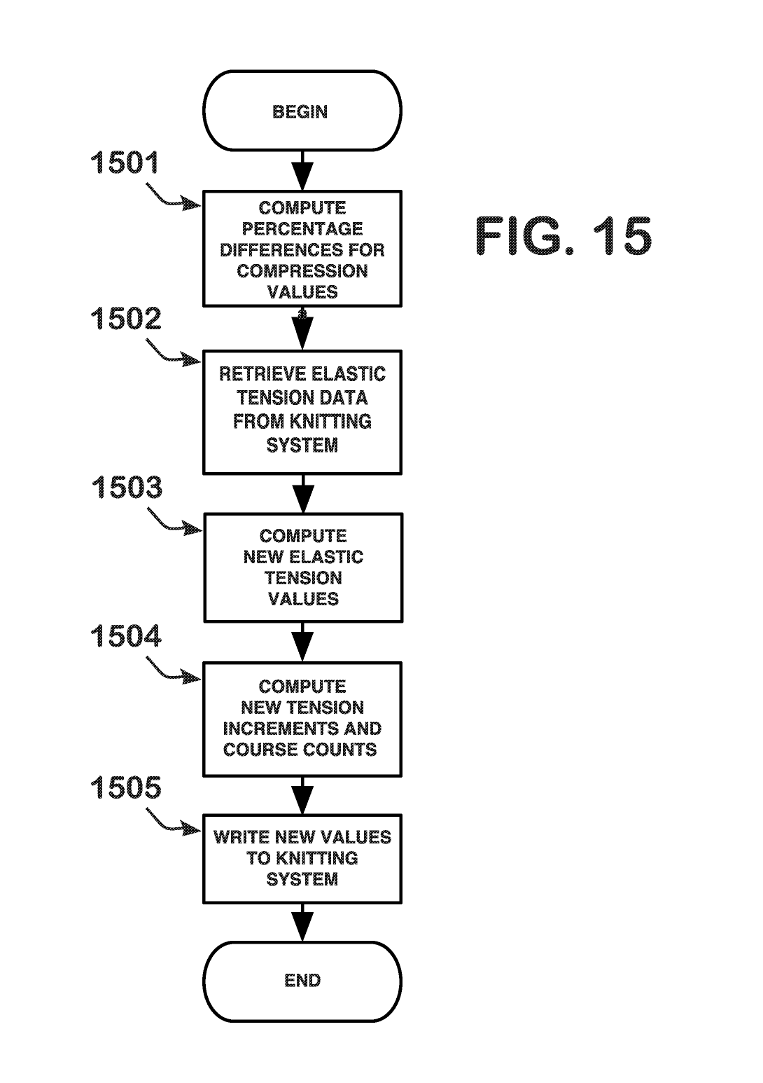

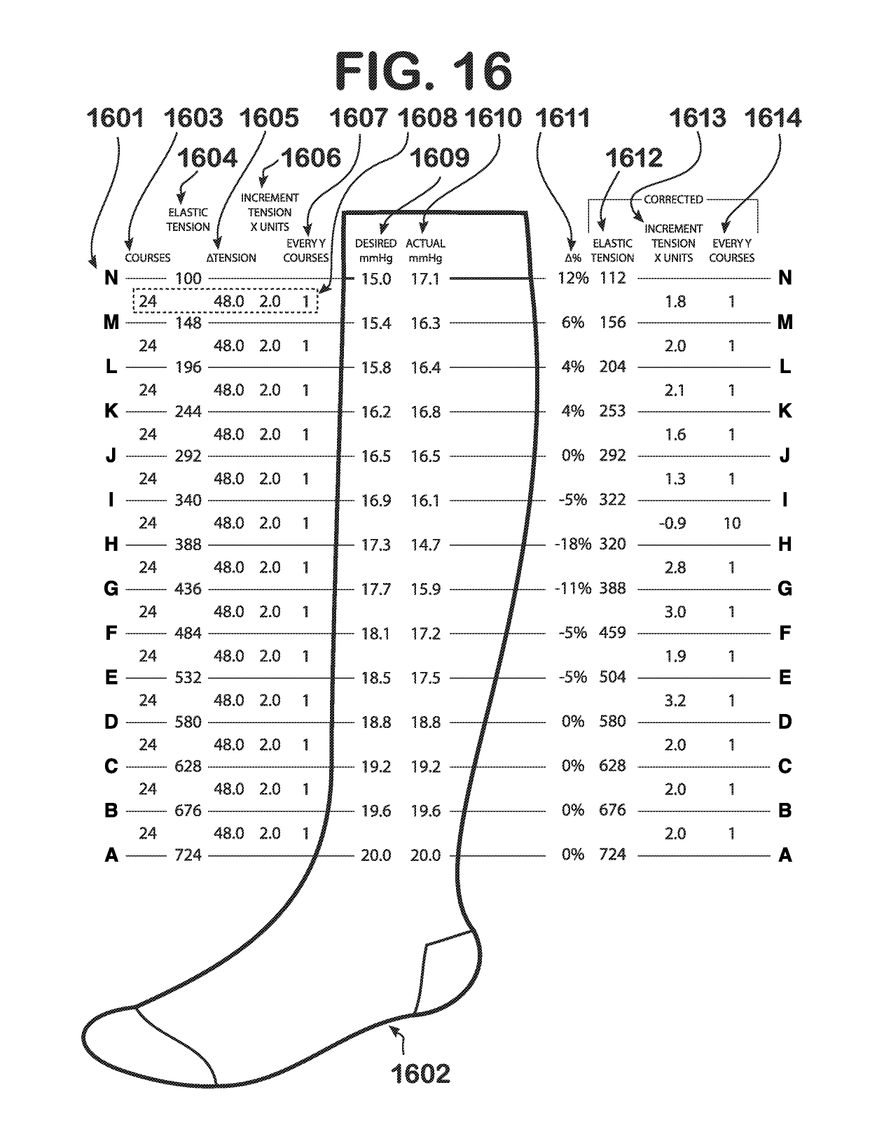

FIG. 15 is a flowchart of the process, and FIG. 16 presents sample data typical of that used to calculate correcting factors for a system knitting a compression garment. The compression garment to be measured is created by a knitting program, familiar to anyone skilled in this art, divided into program segments, each of which specifies a section of garment corresponding in width to one sensor unit in the measurement array. In our sample data, each sensor unit A through N 1601 corresponds to a portion of the garment, shown in outline 1602 to assist in visualizing the relationship of each sensor unit to a position in the garment. Each segment is 24 courses in length 1603. The knitting program will establish an elastic tension setting for each segment, here shown in numerical units 1604. Those skilled in this art will recognize that these units are determined by the specific knitting hardware and yarn feeder employed, and that the units here are provided for illustrative purposes only. The change in tension units between each segment can be seen 1605. As is typical in a knitting program, the instructions to the knitting system controller are given as the number of tension units to add or subtract 1606 from the current setting at a point in the knitting process, and the number of courses between adjustments 1607. For instance, in the segment of the garment between measurement point N and M 1608, the compression must be adjusted by 48 units in 24 courses. To do so, the knitting program is instructed to increment the tension by 2 units for every course.

The calculations for the process of modifying the compression data for the knitting system are performed by the software control application for the invention. To begin, the application computes the percentage difference 1611 between each actual compression data value as measured by the invention 1610 and the desired value 1609. The latter two values are known from the set-up and measurement process. A new elastic tension setting 1612 may be calculated from the percentage difference, and using that setting the corresponding tension increment and course count for the segment may be calculated.

A person skilled in this art will recognize that the calculations required by a given knitting system may be more complex than those shown here. For instance, the relationship between elastic tension and the calculated percentage change in compression may by nonlinear, and may require the application of one or more correction factor for portions of the compression range. In addition, the adjustment of elastic tension may be only one of several changes required by the knitting software. Similar calculations can be performed for the cylinder height and cam settings of the knitting system. The specific calculations for knitting given knitting system may be programmed into the control application, and the application may require the creation of additional interface displays to allow the operator to change the parameters for those calculations.

Returning to the flowchart of FIG. 15, the process of modifying the compression parameters used by the knitting system begins with the calculation of the percentage differences between the desired and actual compression values 1501 by the control application. The application then retrieves the elastic tension data from the knitting system 1502. New tension values are computed 1503, modified tension increments and course counts are calculated 1504, and the new values are written to the knitting system 1505. All communication between the control application and knitting system occur over the network connections illustrated in FIG. 6A.

Turning to FIG. 13D, we see an illustration of the computer tablet measurement interface, after the data discussed above has been transferred to the knitting system and a new garment has been manufactured using the modified data values. The series of data points 1321 indicate the compression measurements for the new garment are within the desired tolerances.

The above disclosure sets forth a number of embodiments of the present invention described in detail with respect to the accompanying drawings. Those skilled in this art will appreciate that various changes, modifications, other structural arrangements, and other embodiments could be practiced under the teachings of the present invention without departing from the scope of the present invention.

We claim:

1. A system for measuring pressure in compression garments comprising:
a support structure; and
a plurality of sensor units with the assembly of the support structure and sensor units defining a three-dimensional simulated anatomical form circumferentially stretching at least a portion of the compression garment upon insertion of the assembly into a compression garment, and each sensor unit having a pressure sensor measuring the pressure exerted by the compression garment on the sensor unit.

2. The system of claim 1 wherein the sensor units are supported at intervals along the support structure and extend circumferentially around the support structure.

3. The system of claim 1 wherein the support structure comprises a support rod, and wherein the sensor units are stacked along the support rod.

4. The system of claim 1 wherein the sensor units are disks with predetermined shapes simulating a series of cross-sections of a three-dimensional anatomical form.

5. The system of claim 1 wherein the support structure simulates a three-dimensional anatomical form for insertion into a compression garment; and the sensor units extend circumferentially around the support structure to stretch at least a portion of the compression garment upon insertion.

6. The system of claim 1 wherein at least one sensor unit further comprises a fluid-filled tube extending around the periphery of the sensor unit in contact with the compression garment, and wherein the pressure sensor measures the circumferential pressure exerted by the compression garment on the tube.

7. A system for measuring pressure in compression garments comprising:
a plurality of sensor units; and
a support structure interconnecting the sensor units to define a three-dimensional simulated anatomical form for insertion into a compression garment, with said sensor units circumferentially stretching at least a portion of the compression garment upon insertion, each sensor unit having a pressure sensor measuring the pressure exerted by the compression garment on the sensor unit.

8. The system of claim 7 wherein at least one sensor unit further comprises a fluid-filled tube extending around the periphery of the support structure in contact with the compression garment, and wherein the pressure sensor measures the circumferential pressure exerted by the compression garment on the tube.

9. A system for measuring pressure in compression garments comprising:
a support structure; and
a plurality of sensor units stacked by the support structure to create a three-dimensional simulated anatomical form for insertion into a compression garment, with said sensor units circumferentially stretching at least a portion of the compression garment upon insertion, each sensor unit having a pressure sensor measuring the pressure exerted by the compression garment on the sensor unit.

10. The system of claim 9 wherein the support structure comprises a support rod, and wherein the sensor units are stacked along the support rod.

11. The system of claim 9 wherein the sensor units comprise disks with predetermined shapes simulating a series of cross-sections of a three-dimensional anatomical form.

12. The system of claim 9 wherein at least one sensor unit further comprises a fluid-filled tube extending around the periphery of the sensor unit in contact with the compression garment, and wherein the pressure sensor measures the circumferential pressure exerted by the compression garment on the tube.
